(19) United States
(12) Patent Application Publication
(10) Pub. No.: US 2014/0235948 A1
(43) Pub. Date: Aug. 21, 2014

(54) METHOD FOR SINGLE-FIBER MICROSCOPY USING INTENSITY-PATTERN SAMPLING AND OPTIMIZATION-BASED RECONSTRUCTION

(71) Applicant: The Board of Trustees of the Leland Stanford Junior University, Palo Alto, CA (US)

(72) Inventors: Reza Nasiri Mahalati, Menlo Park, CA (US); Ruo Yu Gu, Palo Alto, CA (US); Joseph M. Kahn, San Carlos, CA (US)

(73) Assignee: The Board of Trustees of the Leland Stanford Junior University, Palo Alto, CA (US)

(21) Appl. No.: 14/182,940

(22) Filed: Feb. 18, 2014

Related U.S. Application Data
(60) Provisional application No. 61/766,432, filed on Feb. 19, 2013.

(51) Int. Cl. A61B 1/07

(52) U.S. Cl. CPC A61B 1/07
USPC 600/160

(57) ABSTRACT

A method for imaging an object with resolution that exceeds the number of spatial modes per polarization in a multimode fiber is disclosed. In some embodiments, the object is interrogated with a plurality of non-spot-sized intensity patterns and the optical power reflected by the object is detected for each intensity pattern. The plurality of optical power values is then used in a non-local reconstruction based on an optimization approach to reconstruct an image of the object, where the image has resolution up to four times greater than provided by prior-art multimode fiber-based imaging methods.

### CROSS REFERENCE TO RELATED APPLICATIONS

This application claims the benefit of U.S. Provisional Application Ser. No. 61/766,432, filed Feb. 19, 2013, entitled “Random Pattern Sampling and Optimization-Based Reconstruction In Single-Fiber Microscopy,” (Attorney Docket 146-036PR1), which is incorporated herein by reference. If there are any contradictions or inconsistencies in language between this application and the case that has been incorporated by reference that might affect the interpretation of the claims in this case, the claims in this case should be interpreted to be consistent with the language in this case.

### FIELD OF THE INVENTION

The present invention relates to imaging in general, and, more particularly, to single-fiber microscopy and endoscopy.

### BACKGROUND OF THE INVENTION

A conventional flexible fiber-based microscope, such as an endoscope, typically includes a bundle containing thousands of optical fibers, a high-power light source, and a miniature camera. The optical fibers in the fiber bundle channel light to the objective end to illuminate a region of interest and relay optical images from the sample end to the camera.

Unfortunately, due to the large number of optical fibers required, these systems are bulky and have a relatively large diameter. As a result, they are incompatible for some applications. When used, the large diameter can give rise to procedural complications and/or patient discomfort. Further, due in part to the limited number of optical fibers in the optical fiber bundle, the image quality of such endoscopes is limited. As a result, efforts toward reducing the size of these imaging systems have been of great interest.

Recently, microscopic imaging using a single multi-mode optical fiber has been demonstrated. The use of multi-mode optical fibers for imaging or analog image transmission has long been of fundamental interest. As a result, single-optical-fiber-based imaging systems are now being pursued vigorously for applications such as endoscopic in-vivo imaging.

Prior-art methods for imaging through a multi-mode optical fiber typically include forming a spot of light in the optical fiber output plane and scanning it through a sequence of locations to sample an object—sometimes referred to as “spot scanning” or “localized sampling.” An image of the sampled object is then obtained via simple local reconstruction. Unfortunately, the number of independently resolvable image features of the object is limited to the total number of spatial modes, per polarization, that propagate through the optical fiber.

A recently demonstrated alternative prior-art method for obtaining an image of an object samples the object using random speckle patterns. The image is then reconstructed using turbid lens imaging techniques. Because this alternative method treats the high-spatial-frequency features of speckle as noise that must be smoothed out, the number of resolvable features is still limited to the total number of spatial modes, per polarization, that propagate through the optical fiber, however.

A method for imaging an object via a single-mode optical fiber, wherein image resolution is improved beyond that achievable with prior-art methods would be a significant advance in the state of the art.

### SUMMARY OF THE INVENTION

The present invention enables imaging using one multi-mode optical fiber, wherein the number of resolvable object features exceeds the number of spatial modes propagating through the optical fiber. As a result, embodiments of the present invention can achieve an image resolution several times greater than can be achieved with prior-art imaging methods. Embodiments of the present invention are particularly well suited for use in in-vivo biological imaging applications, such as endoscopy.

An illustrative embodiment of the present invention is a method for imaging an object via a sole multi-mode optical fiber. In the method, non-local reconstruction, based on an optimization-based reconstruction technique, is used to increase the number of resolvable features beyond the number of optical modes propagating through the optical fiber. In some embodiments, the present invention enables the number of resolvable features to equal at least four times the number of optical modes propagating through the optical fiber.

In some embodiments of the present invention, an object is imaged via an imaging system comprising a spatial light modulator that excites a sequence of different superpositions of modal fields in a multi-mode optical fiber. At the output of the optical fiber, these generate a sequence of intensity patterns that are used to interrogate the object. The modal fields are mixed due to squaring inherent in field-to-intensity conversion, which enables a description of the output intensity patterns using modes of higher order than the fields propagating through the optical fiber. Light reflected from the object is coupled back into the optical fiber and detected. An image of the object is then reconstructed based on the detected light using an optimization-based reconstruction technique, such as linear optimization, convex optimization, and the like.

In some embodiments, the imaging system is calibrated to determine a set of spatial light modulator patterns suitable for producing a sequence of spots on a grid of positions in the output plane of the optical fiber. In some embodiments, a transfer matrix is generated that maps each pixel of the spatial light modulator and each pixel of a camera that measures the output intensity pattern of the optical fiber. This transfer matrix enables direct computation of the set of spatial light modulator patterns suitable for giving rise to a set of intensity patterns for interrogating an object.

In some embodiments, a sequence of random pixel patterns at the spatial light modulator are used to create a sequence of random field patterns at the output of the optical fiber, which give rise to a sequence of random intensity patterns used to interrogate the object. The light reflected by the object for each of the random intensity patterns is used to reconstruct an image of the object using an optimization-based reconstruction technique.

In some embodiments, a plurality of designed intensity patterns is used to interrogate an object. Each of the designed intensity patterns is developed based on a specific desired illumination pattern at the object.

An embodiment of the present invention is a method for imaging an object, the method comprising: (1) for i=1 through M; (a) interrogating the object with a first intensity pattern, IPᵢ; (b) determining the intensity of a reflected signal, RSᵢ, where RSᵢ includes a portion of IPᵢ that is reflected from the object; and (c) assigning a value to element pᵢ based on the intensity of RSᵢ; (2) forming a first vector that includes elements p₁ through p_{M}; and (3) reconstructing an image of the object via an optimization-based reconstruction technique that is based on the first vector.

Another embodiment of the present invention is a method for imaging an object, the method comprising: providing a plurality of field patterns at a first facet of a multimode optical fiber; interrogating the object with a plurality of intensity patterns, each of the plurality of intensity patterns being generated at a second facet of the multimode optical fiber, wherein each of the plurality of intensity patterns is based on a different field pattern of the plurality thereof; detecting a plurality of power values, wherein each of the plurality of power values is based on (1) light reflected from the object for a different intensity pattern of the plurality thereof and (2) a characteristic of the object; and reconstructing an image of the object based on an optimization-based reconstruction using the plurality of power values.

Yet another embodiment of the present invention is a method for imaging an object, the method comprising: reflecting a first light signal from a spatial light modulator as a second light signal; controlling a pixel pattern of a spatial light modulator to generate a plurality of field patterns at a first facet of a multimode optical fiber; interrogating the object with a first plurality of intensity patterns, wherein each of the first plurality of intensity patterns is based on a different field pattern of the plurality thereof; detecting a plurality of power values, wherein each of the plurality of power values is based on (1) light reflected from the object for a different intensity pattern of the first plurality thereof and (2) a characteristic of the object; and reconstructing an image of the object based on an optimization-based reconstruction using the plurality of power values.

### BRIEF DESCRIPTION OF THE DRAWINGS

FIG. 1 depicts a schematic diagram of a portion of an imaging system in accordance with an illustrative embodiment of the present invention.

FIG. 2A depicts the intensity of a spot formed during a calibration of a prior-art spot-scanning system.

FIG. 2B depicts an intensity pattern in accordance with the present invention. As discussed below, intensity pattern 204 can be either a designed intensity pattern or a random intensity pattern.

FIG. 3 depicts operations of a method for imaging an object in accordance with the illustrative embodiment of the present invention.

FIG. 4A depicts sub-operations suitable for calibrating system 100 for use with a sequence of random intensity patterns.

FIG. 4B depicts sub-operations suitable for calibrating system 100 for use with a sequence of designed intensity patterns.

FIG. 5 depicts a comparison of normalized singular value magnitudes of optimization-based reconstruction using random intensity patterns and designed intensity patterns.

FIG. 6 depicts a comparison of PSF for localized reconstruction versus optimized reconstruction.

FIG. 7 depicts singular values of electric-field patterns at facet 130 and corresponding intensity patterns at target position 152 of system 100 in accordance with the present invention.

### DETAILED DESCRIPTION

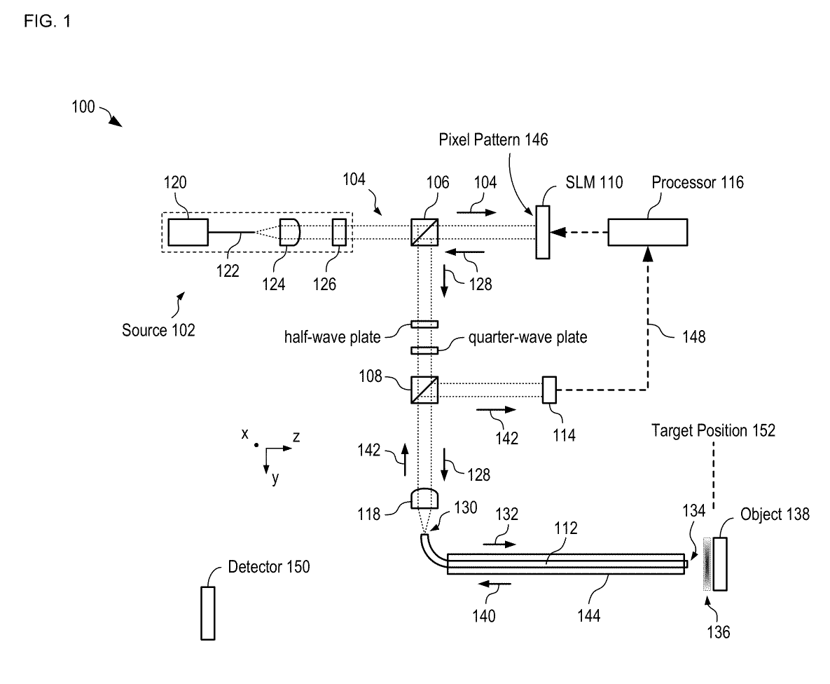

FIG. 1 depicts a schematic diagram of a portion of an imaging system in accordance with an illustrative embodiment of the present invention. Imager 100 includes source 102, conventional beam splitters 106 and 108, SLM 110, optical fiber 112, power monitor 114, processor 116, and lens 118. Imager 100 is operative for interrogating object 138 with a series of intensity patterns, whose configurations are controlled by SLM 110.

Source 102 includes laser 120, polarization-maintaining, single-mode optical fiber 122, collimator 124, and linear polarizer 126. Laser 120 emits 1550-nm light, which is coupled through polarization-maintaining, single-mode optical fiber 122 to collimator 124. Collimator 124 collimates the light, which passes through linear polarizer 126 as beam 104. One skilled in the art will recognize that the desired wavelength of beam 104 depends on the application for which imager 100 is intended.

Spatial-light modulator (SLM) 110 is a phase-only nematic liquid-crystal-on-silicon (LCOS) spatial-light modulator that includes a 256×256 array of pixels. Each approximately square pixel is approximately 18 microns on a side. Each pixel in SLM 100 can be controlled to give rise to a phase change on incident light within the range of 0 to 2π with 5-6 bit resolution. The switching speed of each pixel (0 to 2π, 10%-90% rise or fall time) is approximately 50 milliseconds. Some embodiments include an amplitude-only SLM. Some embodiments include a phase-and-amplitude SLM. The relative phases of pixels collectively define the configuration of SLM 110 (i.e., pixel pattern 146).

It will be clear to one skilled in the art, after reading this Specification, that the device characteristics of SLM 110, such as device size, array size, pixel type, and pixel dimension, are matters of design and are typically based on the application for which system 110 is intended and that SLM can have any practical device characteristics without departing from the scope of the present invention.

Optical fiber 112 is a multi-mode optical fiber suitable that supports N modal fields at the wavelength of optical signal 104. An exemplary optical fiber 112 is a parabolic-index, multimode optical fiber having a 50-micron diameter core, a length of one meter, and an NA of 0.19 that supports 45 modes (i.e., N=45) at a wavelength of 1550 nm. It will be clear to one skilled in the art, after reading this Specification, that optical fiber 112 can have any suitable characteristics, such as core diameter, length, NA, or number of supported modes. In some embodiments, optical fiber 112 is a step-index multimode optical fiber.

Power monitor 114 is a conventional power monitor whose output signal indicates the amount of optical power it receives. Power monitor 114 provides output signal 148 to processor 116.

Processor 116 is a conventional processor capable of providing control signals to SLM 110, as well as receiving output signals from power monitor 114 and reconstructing an image of object 138 based on these output signals.

In operation, beam 104 is directed to SLM 110 via conventional beam splitter 106.

Processor 116 controls pixel pattern 146 to impart a field pattern on beam 104, which is reflected by SLM 110 as beam 128. Beam 128 is directed to optical fiber 112 by beam splitters 106 and 108 and coupled into facet 130 of optical fiber 112 via conventional lens 118.

The field pattern of beam 128 at facet 130 stimulates a pattern of the N modal fields in optical fiber 112, which collectively define light signal 132. At facet 134, each of the fiber modes exits as a beam and these beams collectively give rise to intensity pattern 136 at target position 148. It should be noted that a quarter-wave plate and half-wave plate can be optionally included in the free-space path of beam 128 (typically between beam splitters 106 and 108) to mitigate polarization effects on intensity pattern 132.

Optical fiber 112 is typically contained within rigid sleeve 144, which restricts motion of the optical fiber to mitigate perturbation of the pattern of optical modes once the optical fiber has been calibrated and/or during operation of system 100.

Object 138 reflects a portion of intensity pattern 136 back into facet 134 as light signal 140. The amount of light reflected by object 138 is dependent upon the configuration of the intensity pattern 136 and the reflective characteristics of the object.

At facet 130, light signal 140 is launched into free space as beam 142, which is collimated by lens 118. Beam splitter 108 redirects beam 142 to power monitor 114, which provides an intensity value to processor 116.

By interrogating object 138 with a sequence of different intensity patterns and monitoring the reflected intensity, as discussed below, system 100 enables reconstruction of a complete image of object 138.

### Prior-Art Multimode-Optical Fiber Imaging Methods

It will be instructive, prior to discussing methods in accordance with the present invention, to present prior-art methods for imaging an object using a multimode optical fiber.

Imaging systems similar to system 100 have previously been used to image objects using a method commonly referred to as “spot scanning,” as disclosed by I. N. Papadopoulos, et al., in “Focusing and scanning light through a multimode optical fiber using digital phase conjugation,” in Lab Chip 20, pp. 10582-10590 (2012), S. Bianchi, et al., in “A multi-mode optical fiber prove for holographic micromanipulation and microscopy,” Lab Chip 12, pp. 635-639 (2012), and T. Cizmar et al., in “Exploiting multimode waveguides for pure optical fiber-based imaging,” Nat. Commun. 3, pp. 1-9 (2012).

In a conventional spot-scanning method, an SLM is used to form a sequence of localized intensity patterns (i.e., spots) on an object, where a sequence of pixel patterns on the SLM gives rise to a light spot located at a different position on a “grid” of M positions on the object. The M pixel patterns corresponding to each grid position are first determined using a calibration procedure, wherein a camera is typically used at the output of the multimode optical fiber, and the SLM pattern is optimized iteratively to form a spot at each of the desired M positions. The amount of power reflected from the object while the spot is at each grid position is then measured.

In an alternative prior-art spot-scanning method, a transfer matrix between the pixel pattern of the SLM and the desired grid positions is determined by monitoring spot position using a camera. Once the transfer matrix of the multimode fiber is known, the M SLM patterns suitable for forming a spot at each of the M grid positions can be computed directly.

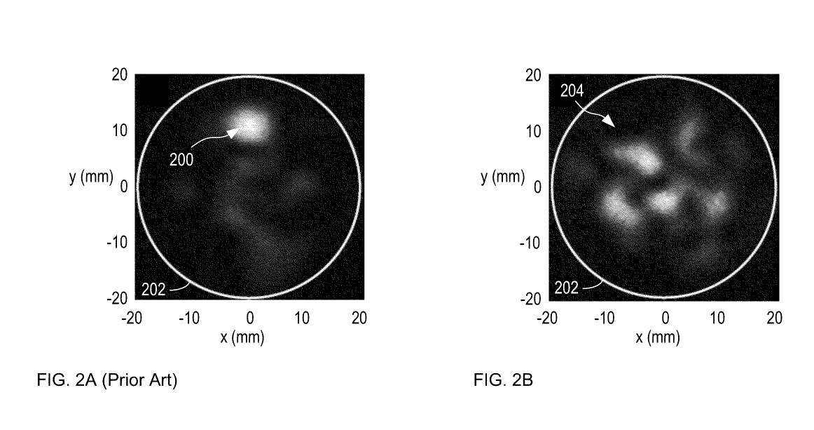

FIG. 2A depicts the intensity of a spot formed during a calibration of a prior-art spot-scanning system. Spot 200 is formed via an imaging system analogous to system 100 described above. Region 202 denotes the area within which spots can be generated. While substantially all of the optical energy within region 202 is included in spot 200, it can be seen from the figure that there are some stray regions of optical energy within the region. Typically, these stray regions do not contribute significantly to the detected reflected signal from an object and can be ignored.

Using these methods, once the M SLM patterns are defined, the object is placed at the output of the multimode optical fiber. When the ith intensity pattern I_{out,i}(x,y) is displayed at the multimode optical fiber output, the reflected power coupled back into the optical fiber is given by:

pᵢ≈k∫∫I_{out,i}(x,y)R_{obj}(x,y)dxdy, (1)

where R_{obj}(x,y) is the object reflectivity and k is a coupling coefficient.

Once each grid position at the object has been sampled, an image, W(x,y), of the object is estimated using local reconstruction techniques from the M power values, where:

W(x,y)=Σᵢ₌₁^{M}pᵢsᵢ(x,y), (2)

where sᵢ(x,y) is unity for (x,y) inside the ith pixel and zero otherwise. The ith pixel is centered at (xᵢ,yᵢ), the centroid of I_{out,i}(x,y).

It should be noted that, in local sampling and reconstruction, the number of resolvable image features cannot exceed the number of mutually orthogonal intensity patterns that can be formed at the MMF output. Further, the number of mutually orthogonal intensity patterns cannot exceed the number of modes N and the number of resolvable image features approximately equals the number of modes N. It is known, however, that forming a satisfactory image of N features requires sampling using M≧4N localized intensity patterns.

The use of conventional local sampling and reconstruction techniques, as described by equations (1) and (2), provides a point-spread function (PSF) proportional to I_{out,i}(x,y), if it is assumed that M>>N. In a graded-index multimode optical fiber, the PSF shape and width varies as a function of the spot centroid (xᵢ,yᵢ)—it is narrowest at the center of the output plane, where, in the limit of many modes N, it ideally approaches a diffraction-limited Airy disk:

$${I}_{A} ( η r ) = {{I}_{o} ( \frac{2 {J}_{1} ( η r )}{η r} )}^{2} , \qquad ( 3 )$$

where r=\sqrt{x²+y²}, η=2πNA/λ, and Iₒ is a normalization constant. It should be noted that the ideal PSF in Eq. (3) depends only on λ/NA and not on N, and has a peak-to-zero width of 0.61λ/NA and half-width at half-maximum (HWHM) of 0.26λ/NA.

It is an aspect of the present invention that, as compared to using spot-scanning and local reconstruction, improved imaging of an object can be achieved by sampling the object with a sequence of intensity patterns and reconstructing the image via an optimization-based reconstruction technique. Optimization-based reconstruction techniques in accordance with the present invention include, without limitation, linear optimization, convex optimization, and the like. Further, the use of methods in accordance with the present invention enable image resolution that is up to four times better than can be achieved with prior-art imaging methods.

Multimode-Optical Fiber Imaging Methods in Accordance with the Present Invention

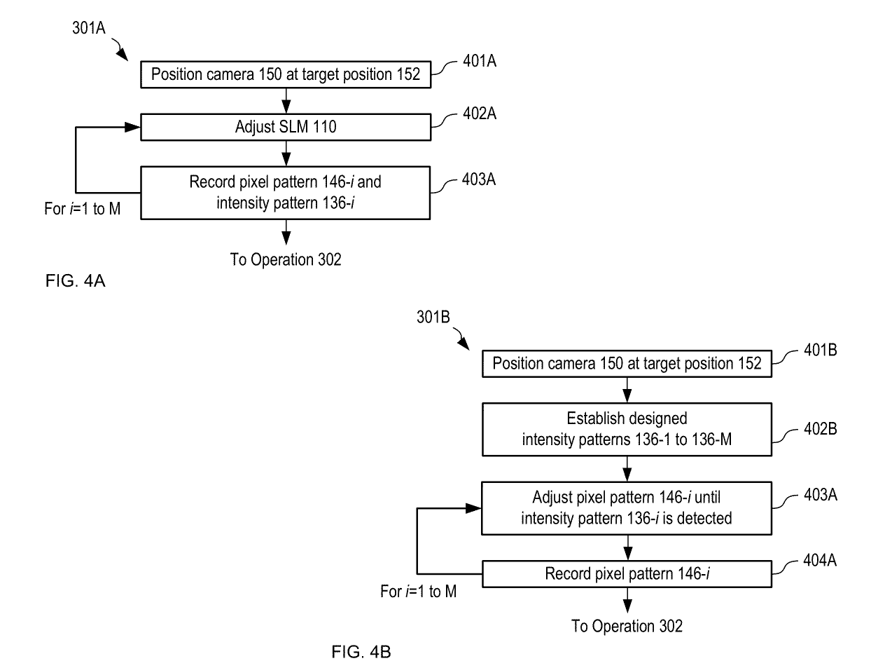

In contrast to prior-art imaging methods, the present invention interrogates an object using a plurality of intensity patterns and reconstructs an image of the object using optimization-based reconstruction. Intensity patterns in accordance with the present invention include spots, as described above and with respect to spot-scanning, as well as non-spot-shaped patterns of optical energy. In some embodiments of the present invention, intensity patterns are “random intensity patterns.” In some embodiments, the intensity patterns are “designed intensity patterns.” Random and designed intensity patterns are discussed below and with respect to FIGS. 4A-B.

FIG. 2B depicts an intensity pattern in accordance with the present invention. As discussed below, intensity pattern 204 can be either a designed intensity pattern or a random intensity pattern.

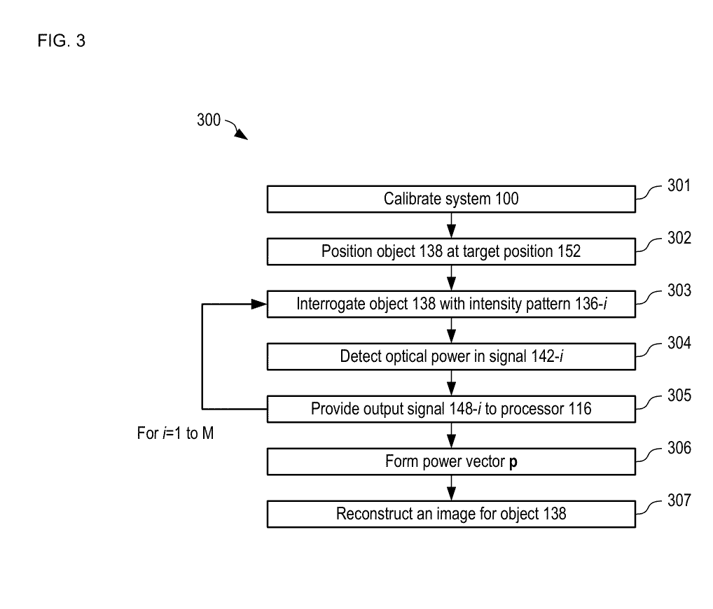

FIG. 3 depicts operations of a method for imaging an object in accordance with the illustrative embodiment of the present invention. Method 300 begins with operation 301, wherein system 100 is calibrated to develop a sequence of intensity patterns suitable for interrogating object 138.

Imaging with Random Intensity Patterns

FIG. 4A depicts sub-operations suitable for calibrating system 100 for use with a sequence of M random intensity patterns. Operation 301A begins with sub-operation 401A, wherein detector 150 is located at target position 152. Suitable detectors for use in operation 301A include, without limitation, phosphor-coated CCD cameras, focal plane arrays of suitable detectors, and the like. In some embodiments, intensity pattern 136 is magnified prior to imaging it onto detector 150.

At sub-operation 402A, for each of i=1 through M, processor 116 adjusts SLM 110 to display pixel pattern 146-i, where the pixel pattern is a “random pixel pattern.”

A random pixel pattern is generated at SLM 110 by grouping the pixels of the SLM into blocks of 8×8 pixels, with the phase piecewise-constant over a block. The pixel patterns are referred to as “random” because each block is independently assigned a phase within the range of 0 to 2π with uniform probability over that range. As a result, a random pixel pattern has no intentional correlation to any other pixel pattern.

The random pixel pattern at SLM 110 gives rise to a random field pattern at facet 130. A random field pattern is a field of optical energy having a plurality of regions within it, where the phase and amplitude of each region are dependent on a random pixel pattern from an SLM.

As discussed above, the field pattern provided to facet 130 excites a collection of modes within optical fiber 112 that give rise to intensity pattern 136-i at target position 152. Since intensity pattern 136-i is based on a random field pattern (and random pixel pattern), intensity pattern 136-i has no correlation to other intensity patterns within the set of M intensity patterns. For the purposes of this Specification, including the appended claims, the term “random intensity pattern” is defined as an intensity pattern produced at a first facet of an optical fiber by a random field pattern provided at a second facet of the optical fiber. Non-random (i.e., designed) pixel patterns, field patterns, and intensity patterns are discussed below and with respect to FIG. 4B.

It will be clear to one skilled in the art, after reading this Specification, that myriad ways to generate appropriate pixel patterns 146 exist and that any practical arrangement of pixels suitable for giving rise to an appropriate intensity pattern 136-i is within the scope of the present invention.

At sub-operation 403A, the calibration procedure is completed by recording pixel pattern 146-i and intensity pattern 136-i at processor 116.

Imaging with Designed Intensity Patterns

Imaging an object with a sequence of random intensity patterns enables image resolution that is four times better than prior-art multimode fiber imaging methods. It is also possible to image an object with a set of intensity patterns that have specific, desired arrangements of optical intensity, such that the intensity patterns interact with the object in a specific manner (i.e., designed intensity patterns). The use of designed intensity patterns enables comparable image resolution as for random intensity patterns. It is an aspect of the present invention, however, that by using designed intensity patterns, system 100 is less sensitive to noise. For the purposes of this Specification, including the appended claims, the term “designed intensity pattern” is defined as an intensity pattern that is designed according to some specified procedure in order to have some desired characteristics, in contrast to a random intensity pattern.

In order to interrogate object 138 with a set of designed intensity patterns, system 100 is first calibrated to develop a sequence of pixel patterns 146 that give rise to the desired sequence of designed intensity patterns.

FIG. 4B depicts sub-operations suitable for calibrating system 100 for use with a sequence of designed intensity patterns. Operation 301B begins with sub-operation 401B, wherein object 138 is replaced by detector 150, as described above and with respect to operation 301A.

At sub-operation 402B, a set of M designed intensity patterns is established.

From the prior art, it is known that every possible intensity at the output of a multimode fiber, Iₒᵤₜ(r,φ), can be decomposed, in polar coordinates, into the intensity modes \tilde{E}ₗₘ(r,φ):

Iₒᵤₜ(r,φ)=Σ_{0≦j≦4N}\tilde{b}ⱼ\tilde{E}ⱼ(r,φ).

In some embodiments, each I_{out,i} is first chosen to minimize noise amplification during image reconstruction, using:

I_{out,i}(r,φ)=|Σ_{0≦k≦4N}b_{k,i}Eₖ(r,φ)|².

where the coefficients b_{k,i} are:

$${b}_{k , i} = {arg min}_{{b}_{k , i}} {\sum }_{0 \leq j \leq 4 N} \vert {δ}_{ji} - {\int \int }_{fiber core} {\tilde{E}}_{j}^{*} ( r , φ ) \vert {\sum }_{0 \leq k \leq 4 N} {b}_{k , i} {E}_{k} ( r , φ ) {{\vert}^{2} r d r d φ \vert}^{2} .$$

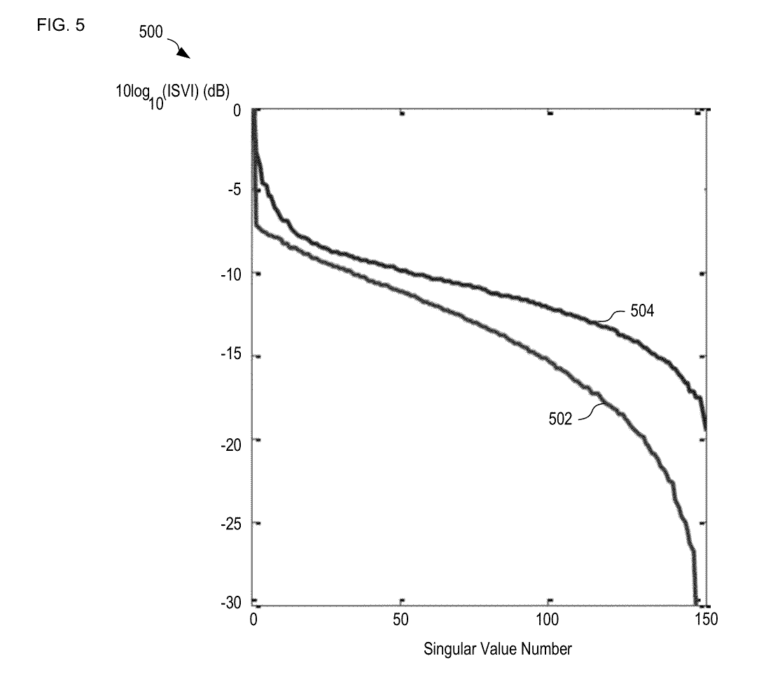

FIG. 5 depicts a comparison of normalized singular value magnitudes of optimization-based reconstruction using random intensity patterns and designed intensity patterns.

Trace 502 denotes singular values based on random intensity patterns, while trace 504 denotes singular values based on designed intensity patterns. A comparison of traces 502 and 504 reveals that the intensity matrix Ĩ has a more equal distribution of singular values than when they are generated randomly

At sub-operation 403B, for each of i=1 through M, processor 116 adjusts SLM 110 until the designed intensity pattern 136-i is detected at detector 150. In some embodiments, the fiber transfer matrix for fiber 112 is first determined. In such embodiments, at sub-operation 403B, the pixel patterns 146 that give rise to the desired sequence of designed intensity patterns can be directly calculated. In some embodiments, the fiber transfer matrix is assumed to be the identity matrix. In such embodiments, the desired intensity mode, \tilde{E}ₖ(r,φ), at fiber facet 132 is generated by providing the same intensity mode, \tilde{E}ₖ(r,φ), fiber facet 130. It should be noted that, since the fiber transfer matrix typically deviates from the identity matrix, the performance of such embodiments is normally slightly degraded.

At sub-operation 404B, pixel pattern 146-i is recorded at processor 116 to complete the calibration procedure.

Returning now to method 300, at operation 302, object 138 is positioned at target position 152.

At operation 303, for i=1 to M, object 138 is interrogated with intensity pattern 136-i.

At operation 304, signal 142 is detected at power monitor 114. The reflected power pᵢ coupled back into fiber 112 is given approximately as described in Equation (1) above. Discretizing the (x,y) plane at target position 152 into a grid of L pixels with spacing Δx=Δy, with the k^{th} pixel centered at (xₖ,yₖ), the integral in Equation (1) can be approximated as the summation:

$${p}_{i} \approx \tilde{κ} {\sum }_{k = 1}^{L} {I}_{out , i} ( {x}_{k} , {y}_{k} ) {R}_{obj} ( {x}_{k} , {y}_{k} ) , \qquad ( 4 )$$

where \tilde{K}=KΔxΔy is the normalized coupling coefficient.

At operation 305, power monitor provides output signal 148-i to processor 116. Output signal 148-i indicates the reflected optical power from object 138 when interrogated with intensity pattern 136-i.

Operations 303 through 306 are repeated M times such that object 138 is interrogated with the full set of intensity patterns developed while system 100 is calibrated at operation 301.

At operation 306, processor 116 forms power vector, p, which is a M×1 vector containing the values of output signals 148-1 through 148-M. The i^{th} entry of p is pᵢ and Ĩ is defined to be an M×L matrix whose i^{th} row is I_{out,i}(xₖ,yₖ).

At operation 307, processor 116 reconstructs an image for object 138. The image is reconstructed based on power vector, p.

In order to reconstruct an image, an image W(x,y) in discretized form W(xₖ,yₖ) is represented as an L×1 vector w, whose k^{th} entry is W(xₖ,yₖ). The reconstructed image ŵ is obtained by solving a linear optimization problem:

$$\hat{w} = {arg min}_{w} {‖ p - \tilde{I} w ‖}_{2} , \qquad ( 5 )$$

where ∥ ∥₂ denotes an I²-norm. Intuitively, ŵ represent the object reflectivity pattern which, if sampled by the intensity patterns Ĩ, would yield samples closest to the observed samples p. Equation (4) can be solved as:

ŵ=VD⁻¹U^{T}p, (6)

where superscript ^{T} denotes matrix transpose and Ĩ=UDV^{T} is the compact singular value decomposition of Ĩ. In some embodiments, a reconstructed image is obtained by minimizing a different norm (e.g., the I¹-norm) of the difference between p and Ĩw.

The image of object 138 is computed using Equation (6), which yields a corresponding Ŵ(xₖ,yₖ), wherein the reconstructed image is Ŵ(x,y)=Σₖ₌₁^{L}Ŵ(xₖ,yₖ)sₖ(x,y), where sₖ(x,y) is unity for (x,y) inside the i^{th} pixel and zero otherwise.

It should be noted that the number of singular values Q corresponds to the number of resolvable image features. For a multimode optical fiber that supports a large number of modes N, the number of resolvable features Q can be as high as 4N. Achieving this resolution requires a number of random intensity patterns and a number of pixels at least that large (i.e., M≧4N and L≧4N).

As discussed above, local reconstruction requires localized spot patterns, so it can only resolve N image features. The fourfold resolution enhancement corresponds to a twofold reduction in the width of the PSF at the center of the fiber output plane.

In a graded-index multimode optical fiber, the PSF shape and width varies as a function of the pixel coordinate (xₖ,yₖ). It is narrowest at the center of the output plane where, in the limit of many modes N, it ideally approaches a diffraction-limited Airy disk:

$${E}_{A} ( 2 η r ) = {E}_{0} \frac{2 {J}_{1} ( 2 η r )}{2 η r} , \qquad ( 7 )$$

where 2η=4πNA/λ and Eₒ is a normalization constant. In similar fashion to Equation (3) above, the ideal PSF in Equation (7) depends only on λ/NA and not on N. Its peak-to-zero width is 0.3λ/NA, precisely half that of Equation (3), while its HWHM is 0.18λ/NA, about 0.69 times that of Equation (3).

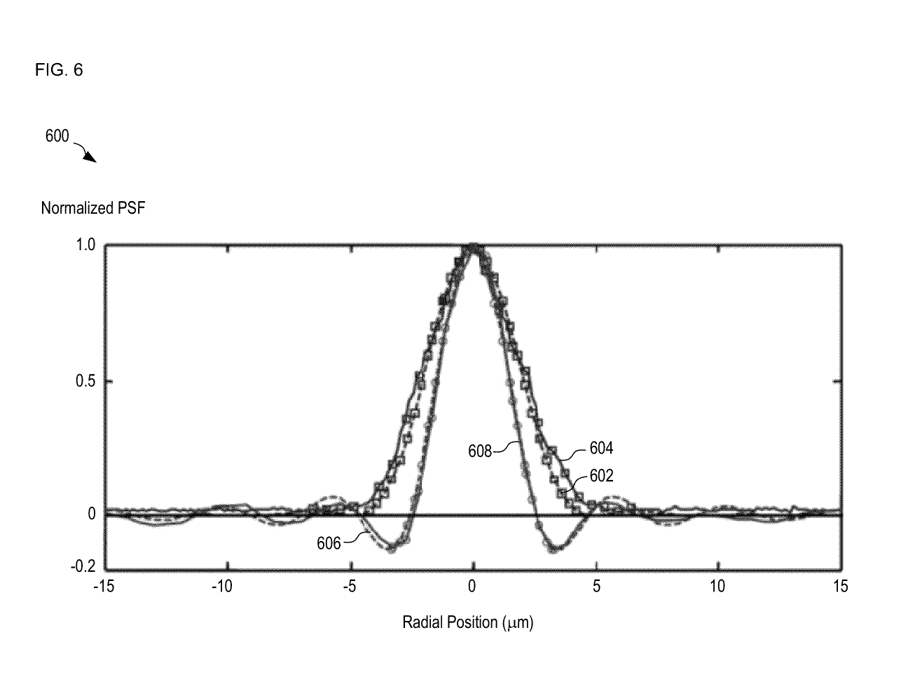

FIG. 6 depicts a comparison of PSF for localized reconstruction versus optimization-based reconstruction. Plot 600 provides calculated and experimental data for an imaging system analogous to system 100.

Plot 602 shows the theoretically optimal PSF using conventional local sampling and local reconstruction. Plot 604 shows an experimentally determined PSF using conventional local sampling and local reconstruction. The theoretical PSF shown in plot 602 has a peak-to-zero width of 5.0 microns and a HWHM of 2.1 microns, while the experimentally measured PSF shown in plot 604 has a HWHM of 2.4 microns (˜14% larger). Plots 602 and 604 show that, when using local reconstruction, the PSF at the center of the optical fiber output plane depends only on λ/NA, and is ideally the same as that of a conventional imaging system with the same λ/NA.

Plots 606 and 608 show a theoretically optimal and estimated PSF, respectively, using intensity pattern interrogation and optimized sampling in accordance with the present invention. The ideal PSF shown in plot 606 has peak-to-zero width of 2.5 microns and HWHM of 1.4 microns. Plot 608 shows an estimated PSF for system 100, where object reflectivity R_{obj}(xₖ,yₖ) is set to unity for k=I and zero otherwise, p is the I^{th} column of Ĩ, and the reconstructed image corresponds to the PSF for an object point at (xᵢ,yᵢ). The estimate shown in plot 608 was produced using 3000 random patterns, where only the strongest 131 singular values were used to minimize the effect of noise.

It is known in the prior art that a graded-index multimode optical fiber with finite core diameter d supports N=(⅛)V²=(⅛)(πdNA/λ)² electric field modes per polarization for large V. Here we consider propagation of a finite but large number of modes N in a fiber having an infinite parabolic index profile. In polar coordinates (r,φ), the modes can be approximated by Laguerre-Gaussian modes. Without loss of generality the modes in the plane z=0 can be considered, allowing z-dependent phase factors to be ignored, giving:

$${E}_{lm} ( r , φ ) = \frac{{c}_{lm}}{{w}_{0}} {( \frac{\sqrt{2} r}{{w}_{0}} )}^{l} {e}^{\frac{- {r}^{2}}{{w}_{0}^{2}}} {L}_{m}^{( l )} ( \frac{2 {r}^{2}}{{w}_{0}^{2}} ) {e}^{il φ} , \qquad ( 8 )$$

where Lₘ^{(l)}(•) is the generalized Laguerre polynomial, w₀=\sqrt{dλ/2πNA} is the mode radius, cₗₘ=\sqrt{2m!/π(l+m)!} is a normalization constant, and 0≦2m+l≦nₘₐₓ=\sqrt{2n}.

Using an SLM, any linear combination of these modes can be generated at the fiber output, so the total output field distribution can be described by:

$$\begin{matrix}{E}_{out} ( r , φ ) = {\sum }_{0 \leq 2 m + l \leq {n}_{max}} {a}_{lm} {E}_{lm} ( r , φ ) \\ = {e}^{\frac{- {r}^{2}}{{w}_{0}^{2}}} {\sum }_{0 \leq 2 m + l \leq {n}_{max}} {{\tilde{a}}_{lm} ( \frac{\sqrt{2} r}{{w}_{0}} )}^{l} {( \frac{2 {r}^{2}}{{w}_{0}^{2}} )}^{m} {e}^{il φ} ,\end{matrix} \qquad ( 9 )$$

where the ãₗₘ can be obtained from the aₗₘ. Since N=nₘₐₓ²/2, the total number of “field modes” N is proportional to the square of the upper limit of summation nₘₐₓ. The output intensity distribution is the squared modulus of Equation (9):

$$\begin{matrix}{I}_{out} ( r , φ ) = {e}^{\frac{- 2 {r}^{2}}{{w}_{0}^{2}}} {\sum }_{0 \leq 2 m + l \leq 2 {n}_{max}} {( \frac{\sqrt{2} r}{{w}_{0}} )}^{l} {( \frac{2 {r}^{2}}{{w}_{0}^{2}} )}^{m} ( {b}_{lm} {e}^{a φ} + {b}_{lm}^{*} {e}^{- a φ} ) \\ = {\sum }_{0 \leq 2 m + l \leq 2 {n}_{max}} {\tilde{b}}_{lm} {\tilde{E}}_{lm} ( r , φ ) ,\end{matrix} \qquad ( 10 )$$

where the bₗₘ can be obtained from the ãₗₘ and the \tilde{b}ₗₘ can be obtained from the bₗₘ. The output intensity distribution in Equation (10) is a linear combination of Laguerre-Gaussian modes with mode radius reduced to w₀/\sqrt{2}. Since the upper limit of summation is 2nₘₐₓ, the total number of “intensity modes” is 4N.

It is an aspect of the present invention that all 4N degrees of freedom can be exploited by the optimization-based reconstruction in Equation (6). Using Equation (4), the vector of reflected powers can be written as p=Ĩr, where r is an L×1 vector representing the object reflectivity values R_{obj}(xₖ,yₖ) in the L pixels. Then Equation (6) takes the form:

ŵ=VD⁻¹U^{T}Ĩr, (11)

which simplifies to:

ŵ=VV^{T}r (12)

Each of the Q rows of V^{T} corresponds to an “intensity mode” of the fiber, recovered from the random intensity pattern matrix Ĩ. The object r is thus projected into the space spanned by linear combinations of Q orthogonal “intensity modes” of the fiber. Neglecting noise, all components of the object corresponding to these Q “intensity modes” appear in the image ŵ with unit gain, while other components are passed with zero gain and do not appear in the image.

Neglecting noise, based on Equation (10), we expect the number of significant singular values of the matrix of field patterns to be approximately N, and the number of significant singular values Q of the matrix of intensity patterns to approach 4N, regardless of whether the patterns are random or represent localized spots.

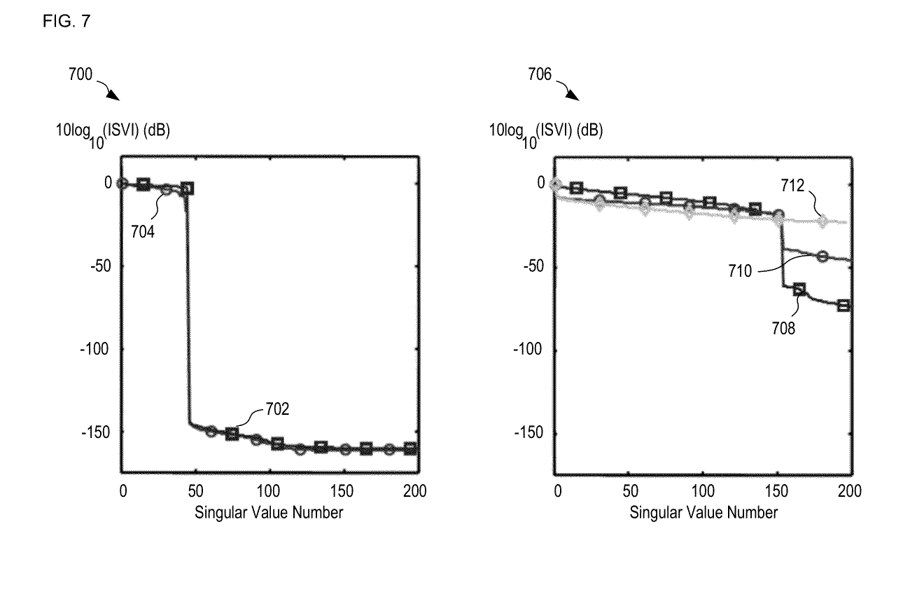

FIG. 7 depicts singular values of electric-field patterns at facet 130 and corresponding intensity patterns at target position 152 of system 100 in accordance with the present invention.

Plot 700 depicts singular values of 500 random electric-field patterns at facet 130 of optical fiber 112. Trace 702 indicates the singular values for electric-field patterns for spot-scanning in accordance with prior-art imaging methods. Trace 704 indicates the singular values for electric-field patterns associated with random intensity patterns in accordance with the present invention.

Plot 706 depicts singular values of 500 random intensity patterns at target position 152. Trace 708 shows simulated singular values of intensity patterns corresponding to the electric-field patterns whose singular values are shown in trace 702 (i.e., spot-scanning-type electric-field patterns). Trace 710 shows simulated singular values of random intensity patterns corresponding to the electric-field patterns whose singular values are shown in trace 704 (i.e., random electric-field patterns). Trace 712 denotes singular values of the random intensity patterns, where the singular values are measured experimentally.

The electric-field patterns shown in each of plot 700 have 45 significant singular values. The corresponding intensity patterns in plot 706 have 153 significant singular values. It should be noted that 153 is the precise number of “intensity modes” obtained by squaring linear combinations of 45 “field modes.” It should be further noted that the singular values shown in plot 706 do not exhibit a sharp drop at 153, presumably because of noise.

It should be noted that a step-index multimode optical fiber supports twice as many modes (at large N) as a graded-index multimode optical fiber; however, step-index multimode optical fibers also exhibit 4N resolvable image features when used in embodiments of the present invention.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A method for imaging an object, the method comprising:
for i=1 through M;
providing a first intensity pattern, IP1ᵢ at a first facet of a multimode optical fiber;
interrogating the object with the first intensity pattern, IP1ᵢ;
determining the power of a reflected signal, RSᵢ, where RSᵢ includes a portion of IP1ᵢ that is reflected from the object; and
assigning a value to element pᵢ based on the power of RSᵢ;
forming a first vector, p, that includes elements p₁ through p_{M}; and
reconstructing a first image of the object by via an optimization-based reconstruction technique that is based on the p.

2. The method of claim 1, wherein each of IP1ᵢ is generated by operations comprising:
providing a field pattern, FPᵢ, at a second facet of an optical fiber;
stimulating a pattern of modal fields in the optical fiber, the pattern of modal fields being based on FPᵢ; and
enabling the pattern of modal fields to generate a second intensity pattern IP2ᵢ at the first facet of the optical fiber, wherein IP1ᵢ is based on IP2ᵢ.

3. The method of claim 2 wherein each field pattern, Fᵢ, is provided by operations comprising:
reflecting a first optical signal from a spatial-light modulator as a second light signal, wherein the spatial-light modulator includes a plurality of pixels; and
controlling the plurality of pixels to provide a pixel pattern, ppᵢ, that produces field pattern FPᵢ at the second facet.

4. The method of claim 3 further comprising calibrating the imager to establish a correlation between each IP1ᵢ and ppᵢ.

5. The method of claim 3, further comprising providing the spatial-light modulator such that at least one pixel is operative for controlling the phase of light reflected from it.

6. The method of claim 1 wherein the first image is reconstructed by operations comprising:
for k=1 through L;
discretizing a first plane that is proximal to the first facet into pixels (xₖ,yₖ); and
computing a second vector, w, according to an optimization relation based on the first vector, p, wherein w includes image values W(xₖ,yₖ), and wherein w represents the first image.

7. The method of claim 1 wherein the first image is reconstructed by operations comprising:
for each of k=1 through L;
discretizing a first plane that is proximal to the first facet into a plurality of pixels (xₖ,yₖ);
discretizing each of first intensity patterns IP1ᵢ through IP1_{M} at each of pixels (xₖ,yₖ) to form discretized intensity patterns IP1′₁ through IP1′_{M}, wherein discretized intensity patterns IP1′₁ through IP1′_{M} collectively define a matrix, Ĩ; and
computing a plurality of image values W(xₖ,yₖ) based on a difference between Ĩw and p, wherein the plurality of image values collectively defines a second vector w that represents the first image.

8. The method of claim 7, wherein the plurality of image values W(xₖ,yₖ) is based on a norm of the difference between Ĩw and p.

9. A method for imaging an object, the method comprising:
providing a plurality of field patterns at a first facet of a multimode optical fiber;
interrogating the object with a plurality of intensity patterns, each of the plurality of intensity patterns being generated at a second facet of the multimode optical fiber, wherein each of the plurality of intensity patterns is based on a different field pattern of the plurality thereof;
detecting a plurality of power values, wherein each of the plurality of power values is based on light reflected from the object for a different intensity pattern of the plurality thereof; and
reconstructing an image of the object based on an optimization-based reconstruction using the plurality of power values.

10. The method of claim 9 further comprising providing the multimode optical fiber as a step-index multimode fiber.

11. The method of claim 9 wherein the linear optimization is based on (1) the I²-norm of the plurality of reflected powers and (2) a vector comprising the plurality of power values.

12. The method of claim 11 wherein the linear optimization comprises operations including minimizing an objective function that is the difference between the I²-norm and the vector.

13. The method of claim 9 further comprising providing each of the plurality of field patterns by operations comprising:
reflecting a first light signal from a spatial light modulator as a second light signal; and
controlling the spatial light modulator to control the field pattern in the second light signal.

14. The method of claim 13 further comprising providing the spatial light modulator such that it comprises an array of pixels, wherein at least one of the pixels is operative for controlling the phase of light reflected from it.

15. The method of claim 13 further comprising providing the spatial light modulator such that it comprises an array of pixels, wherein at least one of the pixels is operative for controlling the intensity of light reflected from it.

16. A method for imaging an object, the method comprising:
reflecting a first light signal from a spatial light modulator as a second light signal;
controlling a pixel pattern of a spatial light modulator to generate a plurality of field patterns at a first facet of a multimode optical fiber;
interrogating the object with a first plurality of intensity patterns, wherein each of the first plurality of intensity patterns is based on a different field pattern of the plurality thereof;
detecting a plurality of power values, wherein each of the plurality of power values is based on light reflected from the object for a different intensity pattern of the first plurality thereof; and
reconstructing an image of the object based on an optimization-based reconstruction using the plurality of power values.

17. The method of claim 16 wherein the optimization-based reconstruction is based on at least one of linear optimization and convex optimization.

18. The method of claim 16 wherein the reconstruction is based on (1) the I²-norm of the plurality of reflected powers and (2) a vector comprising the plurality of power values.

19. The method of claim 16 further comprising:
providing an optical system for interrogating the object with the first plurality of intensity patterns; and
calibrating the optical system by operations including;
displaying a plurality of pixel patterns on the spatial light modulator;
recording a second plurality of intensity patterns at the second facet of the multimode optical fiber, wherein each of the second plurality of intensity patterns is based on a different pixel pattern of the plurality thereof; and
storing the second plurality of intensity patterns as the first plurality of intensity patterns.

20. The method of claim 19, wherein the sequence of random phase patterns are provided by operations comprising:
grouping the pixel pattern into a plurality of pixel regions, each pixel region comprising a plurality of pixels whose phase is piece-wise constant; and
assigning each pixel region a random phase whose probability density is substantially uniformly distributed between 0 and 2π.
